(19) United States
(12) Patent Application Publication
(10) Pub. No.: US 2018/0303144 A1
(43) Pub. Date: Oct. 25, 2018

(54) Nutritional Product for Decreasing Phenylalanine Levels in PKU Patients

(71) Applicant: N. V. Nutricia, Zoetermeer (NL)

(72) Inventor: Amos ATTALI, Utrecht (NL)

(21) Appl. No.: 15/770,444

(22) PCT Filed: Oct. 24, 2016
(86) PCT No.: PCT/NL2016/050729

§ 371 Date: Apr. 23, 2018

(30) Foreign Application Priority Data
Oct. 23, 2015 (NL) PCT/NL2015/050738

(51) Int. Cl. A23L 33/12; A23L 33/155; A61K 31/202; A61K 45/06

(52) U.S. Cl. CPC A23L 33/12; A23L 33/155; A61K 31/202; A61K 45/06; A23V 2002/00; A23V 2200/30; A23V 2250/1626; A23V 2250/1876; A23V 2250/7052; A23V 2250/7058; A23V 2250/706; A23V 2250/708; A23V 2250/712; A23V 2250/304

(57) ABSTRACT

The invention relates to a composition comprising) (i) one or more ω-3 fatty acids selected from the group consisting of DHA, DPA and EPA and (ii) one or more pyrimidine derivatives selected from the group consisting of uridine sources and cytidine sources for use in decreasing or normalizing the phenyl alanine level in the blood of a PKU patient or for use in dietary management of phenylalanine levels in the blood of a PKU patient.

### BACKGROUND OF THE INVENTION

The present invention relates to a composition for use in the treatment of a PKU patient. Further, the invention relates to a nutritional composition comprising protein, lipid and carbohydrates, said composition providing at least all essential amino acids other than phenylalanine.

The genetic mutation characteristic for Phenylketonuria (PKU) impairs the proper functioning of the enzyme phenylalanine hydroxylase (PAH), which normally converts Phenylalanine (Phe) to Tyrosine (Tyr). This mutation causes Phe to accumulate in blood and brain to toxic levels. Additionally, as Tyr is the precursor for the neurotransmitters Dopamine and Noradrenaline, a decrease in Tyr synthesis disrupts the biosynthesis of these catecholamines. In parallel, Phe competes with Tyr and Tryptophan (Trp, the precursor for Serotonin) at amino acid transporters across the blood brain barrier (BBB) and as consequence, high blood Phe concentration also leads to a reduced brain entry of Tyr and Trp, further impacting on their availability for neurotransmitter and protein biosynthesis in neurons.

The availability of Dopamine and Serotonin is critical as these neurotransmitters are involved in a variety of functions, particularly in the Prefrontal Cortex (PFC) which is the main site of higher cognitive functions. In PKU patients still on diet and with plasma Phe concentrations falling within the then recommended levels, metabolism of both Dopamine and Serotonin was proposed to be deficient, as cerebrospinal fluid measurements showed reduced levels of metabolites of these neurotransmitters. In parallel, studies in early and continuously treated children with PKU have demonstrated deficits in executive functioning, including strategic processing, processing speed, problem solving, non-verbal intelligence, working memory and attention flexibility.

Key in the pathophysiology of the PKU is raised Phe levels in the blood. Current treatment in the Netherlands of PKU is characterised by managing these levels between the 120 μmol/l and 360 μmol/l for children (0-12 years) and between 120 μmol/l and 600 μmol/l from 12 years on. Left untreated, Phe levels could raise to 1000 and 2000 μmol/l As patients often have difficulties maintaining the diet, the levels could be anywhere between the previous mentioned levels.

Current nutritional treatment of PKU focuses on a decreased intake of phenylalanine in the diet. Such diet should be taken life long, but is in particular important during childhood and adolescence. Many studies have shown that complete depletion of phenylalanine in the diet is hardly possible. Patients will always want to eat something else than current medical food products based on amino acids as protein source. Knowing that phenylalanine levels in the blood are almost always higher than recommended, the need for a product that could decrease the phenylalanine levels in the blood of PKU patients would solve a real problem in the dietary management of PKU patients.

It is an object of the present invention to provide a composition for use in the treatment of a PKU patient, wherein the composition comprises a combination of two or more active ingredients, which combination of ingredients is effective in reducing or normalizing the phenylalanine level in blood of the patient or effective in maintaining the phenylalanine level in blood within a non-pathological range, also when the PKU patient's diet contains a substantial amount of Phe.

A further object of the present invention is to provide an improved dietary product that can be used for lowering the phenylalanine level in the blood.

### SUMMARY OF THE INVENTION

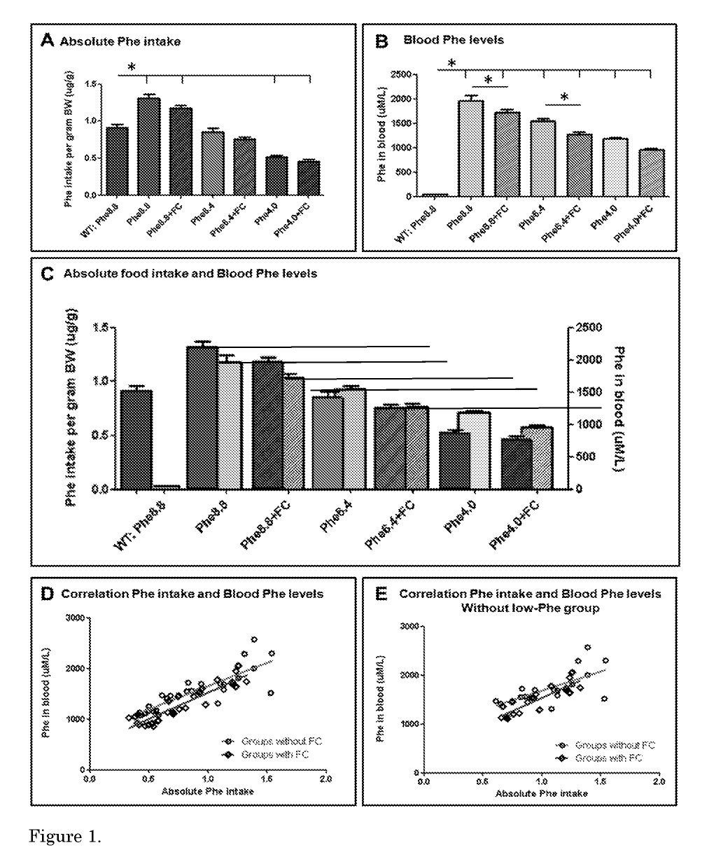

The inventors surprisingly found that a composition comprising an ω-3 fatty acid selected from the group consisting of DHA, DPA and EPA and a pyrimidine derivative selected from the group consisting of uridine sources and cytidine sources, in particular DHA and UMP, was capable of decreasing the phenylalanine concentration in blood. This finding was a totally unexpected result of a study in a scientifically accepted mouse model for PKU. These PKU mice (C57/Bl6^{enu2}) on either a DHA and UMP based diet supplemented with different levels of the amino acid phenylalanine or a control diet without DHA and UMP were compared to wild-type (WT) mice on control diet. Dietary treatment was started at postnatal day 31 and continued for three months. It was surprisingly shown that the PKU mice with DHA and UMP diets were having lower phenylalanine levels in the blood than PKU mice with control diet. (see FIG. 1).

Accordingly, the present invention relates to a composition comprising) (i) one or more ω-3 fatty acids selected from the group consisting of DHA, DPA and EPA and (ii) one or more pyrimidine derivatives selected from the group consisting of uridine sources and cytidine sources for use in decreasing or normalizing the phenylalanine level in the blood of a PKU patient or for use in dietary management of the phenylalanine level in the blood of a PKU patient.

The one or more ω-3 fatty acid selected from the group consisting of DHA, DPA and EPA and/or the one or more pyrimidine derivatives selected from the group consisting of uridine sources and cytidine sources are typically present in the composition for use as active ingredients in accordance with the invention. The composition may comprise one or more further ingredients which may have a beneficial effect with respect to the intended use of the invention, in particular a vitamin B, a methyl donor (such as a choline), a phospholipid or an antioxidant (such as vitamin C, selenium, vitamin E).

The composition may be a pharmaceutical composition containing said active ingredient(s) for use in accordance with the invention and one or more pharmaceutically acceptable excipients. Preferably, the active ingredient(s) for use in accordance with the invention are administered as part of or in combination with a nutritional composition.

Accordingly, the invention further relates to a nutritional composition comprising protein, lipid and carbohydrate wherein the protein provides at least all essential amino acids other than phenyl alanine, and optionally phenylalanine, which—if present—is present in a phenylalanine concentration of 0-10 mg/per gram protein; the lipid comprising one or more ω-3 fatty acids selected from DHA, DPA and EPA in a total concentration of at least 0.05 wt % based on total lipid content; and one or more pyrimidine derivatives selected from uridine sources and cytidine sources. The one or more pyrimidine derivatives selected from uridine sources and cytidine sources are typically present in a total concentration of at least 1 μmol/100 kcal of the nutritional composition. Such nutritional composition is in particular suitable for use in accordance with the invention.

### DETAILED DESCRIPTION OF THE INVENTION

The invention pertains to compositions comprising one or more ω-3 fatty acids, uridine or cytidine or their equivalents, and its use for lowering or normalizing phenylalanine levels in blood of PKU patients.

The term “or”, as used herein, means also “and”, unless specified otherwise or the context dictates otherwise. Hence, “option A or B” means any of the options A, B and A and B.

The term “a” or “an”, as used herein, means “at least one” unless specified other-wise.

When referring to a noun (e.g. a compound, an additive etc.) in singular, the plural is meant to be included, unless specified otherwise.

When referring herein to an acid, e.g. a fatty acid, amino acid, or folic acid, this term is meant to include the conjugated bases of said acid (e.g. folate), salts of the acids and derivatives of the acid of which the body is capable of converting it into the acid (e.g. fatty acid esters, such as triglycerides), unless specified otherwise.

For the purpose of clarity and a concise description, features are described herein as part of the same or separate embodiments, however, it will be appreciated that the scope of the invention may include embodiments having combinations of all or some of the features described.

Protein

The term ‘protein’ is defined as any compound composed of one or more amino acids; the term in particular includes free amino acids, salts thereof, peptides, hydrolysed proteins and intact protein.

PKU patients have to lower the intake of phenylalanine (Phe) in order to keep the Phe levels in blood and brain within a normal range, compared to humans not having PKU. Therefore, in an advantageous embodiment the composition (for use) according to the invention is essential free of phenylalanine or has a relatively low phenylalanine level. Thus, the composition advantageously contains 0-10 mg Phe per gram protein, in particular 0-1 mg Phe per gram protein, preferably 0-0.1 mg Phe per gram protein.

A low level typically means that the content of Phe is lower than in a comparable (nutritional) composition, such as a (nutritional) product of the same type not intended for administration to PKU patients.

In a preferred embodiment, a composition (for use) according to the invention comprises caseino-glyco-macropeptide (cGMP). Preferably complementary essential amino acids other than Phe are added as a protein source in addition to cGMP protein source in order to compensate for the amino acids that are present in low amounts in the GMP protein. The advantage of using cGMP as a protein source for nutritional products is that cGMP has a better taste, a low osmolarity and a balanced amino acid profile, than free amino acids. Further, cGMP is free of Phe or has a low Phe content (e.g., the amino acid sequence of bovine cGMP is free of Phe units, however commercially available cGMP obtained from milk may comprise a trace of Phe from other milk protein components). This makes this protein ideally suited for use in a diet for PKU patients. The protein source preferably comprises about 30% to about 70% by weight of caseino-glyco-macropeptide and about 70% to about 30% by weight of complementary free amino acids.

Preferably, the composition (for use) according to the invention provides an excess of Tyr to compensate for the inability of PKU patients to metabolise Phe into Tyr. Preferably, the Tyr content of a composition (for use) according to the invention is between 7-15 wt. % of the total amino acid content, even more preferably between 8-13 wt. % and even more preferably between 9-12 wt. % of the total amino acid content.

Preferably the protein source provides about 7% to about 80%, more preferably 7-20%, most preferably 7-13% of the dry weight of a composition (for use) according to the invention, In a preferred embodiment suitable for use in an infant formula, the protein source provides about 7% to about 13% of the energy of the composition (for use) according to the invention. Energy percentages can be determined on the basis of generally known contributions of the ingredients of a composition to the caloric value; for protein and digestible carbohydrate the caloric value typically is 4 kcal/gram, for lipids 9 kcal/gram.

Preferably, a nutritional composition (for use) according to the invention contains all essential amino acids other than phenylalanine, i.e. valine, threonine, tryptophan, methionine, leucine, isoleucine, lysine, and histidine. In a further embodiment, the nutritional composition comprises all essential amino acids, including phenyl alanine.

A suitable amino acid profile for a specific type of nutritional composition is known in the art and may be based on known nutritional formulae for PKU patients, e.g. PKU Anamix Junior, PKU Lophlex Advance or PKU Anamix infant (all available from Nutricia Advanced Medical Nutrition).

Further, a nutritional composition (for use) according to the invention may e.g. be based on any of the compositions suitable for PKU patients described in WO 2011/078677, of which the contents with respect to those compositions are incorporated by reference, in particular Tables 2 and 3, with the proviso that a composition (for use) according to the present invention may comprise Phe, and comprises said pyrimidine derivative(s) and said ω-3 fatty acid(s).

Further, a nutritional composition may e.g. be based on any of the compositions suitable for PKU patients described in WO 2013/133711, of which the contents with respect to those compositions are incorporated by reference, in particular Tables 1, 2a and 2b, with the proviso that a composition (for use) according to the present invention may comprise Phe, and comprises said pyrimidine derivative(s) and said ω-3 fatty acid(s).

Further, a nutritional composition may e.g. be based on any of the compositions suitable for PKU patients described in WO 2015/002537, of which the contents with respect to those compositions are incorporated by reference, in particular the compositions of Examples 1-10, with the proviso that a composition (for use) according to the present invention may be free of Phe or may comprise Phe in a lower amount (preferably less than 10 mg/g protein), and comprises said pyrimidine derivative(s) and ω-3 fatty acid(s)

Further, Nestle [Engl.] 2010; 68:58-69 Table 2, of which the contents are incorporated by reference, provides guidelines of daily protein, energy and Phe intake for individuals with PKU.

In particular, a nutritional composition (for use) according to the invention comprises one or more amino acids, preferably at least the essential amino acids other than phenylalanine, more preferably all amino acids in a relative amount given in Table 1.

TABLE 1

| | Preferred |
|---|---|
| | range |
| | g/100 g dry |
| Amino Acids | weight |
| Alanine | 0.5-4.0 |
| Arginine | 1.0-7.0 |
| Aspartic acid | 0.8-6.5 |
| Cysteine | 0.4-3.5 |
| Glutamine | 1.0-5.0 |
| Glycine | 0.8-6.0 |
| Histidine | 0.5-4 |
| Iso-leucine | 0.5-6.0 |
| Leucine | 1.5-10 |
| Lysine | 1-7 |
| Methionine | 0.2-2 |
| Phenylalanine | 0-0.3 |
| Proline | 1-8 |
| Serine | 0.5-5 |
| Threonine | 0.6-6 |
| Tryptophan | 0.2-2.5 |
| Tyrosine | 1.0-9.0 |
| Valine | 1.0-7.0 |

Lipids

The term lipids includes any edible fatty substance generally regarded as fat or oil in food products, including triglycerides, fatty acids and phospholipids.

The total amount of lipids is preferably between 10 and 30 wt. % on dry matter, and/or between 2 and 6 g lipid per 100 ml for a liquid composition.

Long Chain Polyunsaturated Fatty Acid (LCP)

The LCP in a composition (for use) according to the invention comprises at least one LCP selected from docosa-hexaenoic acid (22:6 ω-3; DHA), docosapentaenoic acid (22:5 ω 3; DPA) and eicosa-pentaenoic acid (20:5 ω-3; EPA).

Preferably the present composition contains at least DHA.

Preferably the composition contains DHA and at least one precursor of DHA selected from EPA and DPA, more preferably DHA and EPA. More preferably the present composition comprises DHA, DPA and EPA.

The LCP is preferably provided as one or more compounds selected from the group of triglycerides, diglycerides, monoglycerides, free fatty acids, salts of fatty acids, esters of fatty acids (other than glycerides), phospholipids, lysophospho¬lipids, glycerol ethers, lipoproteins, ceramides, glycolipids. Preferably, the present composition comprises DHA in triglyceride form.

The composition is preferably administered to provide (a total of) 400-5000 mg of the ω-3 fatty acid(s) selected from DHA, EPA and DPA per day, more preferably 500-3000 mg per day, most preferably 1000-2500 mg per day. The present use preferably comprises the administration of DHA, preferably in an amount of 300-4000 mg per day, more preferably 500-2500 mg per day.

An amount per day as described herein means an amount in a daily dosage unit provided by the composition of the invention. Such a daily dosage unit may be a single dosage, but it may also be divided over two or three, or even more daily servings.

The present composition preferably comprises 0.1-40 wt. % DHA based on total fatty acids, preferably 0.3-36 wt. % DHA based on total fatty acids, more preferably 1.0-30 wt. % DHA based on total fatty acids. The present composition preferably comprises 0.05-20 wt. % EPA based on total fatty acids, preferably 0.2-10 wt. % EPA based on total fatty acids, more preferably 0.5-10 wt. % EPA based on total fatty acids. The ratio of the weights of DHA to the sum of EPA and DPA (ω-3) is preferably larger than 1.0, more preferably 1.2-10, more preferably 2-8. The above-mentioned ratios and amounts take into account and optimise several aspects, including taste (too high LCP levels reduce taste appreciation, resulting in a reduced compliance), balance between DHA and precursors thereof to ensure optimal effectiveness in relation to maximum dosage and possibility of product formulations such as liquid form, bar or capsule.

The weight to weight ratio ω-6 fatty acids having at least 20 carbon atoms to ω-3 fatty acids having at least 20 carbon atoms in the present composition is advantageously below 0.5, preferably below 0.2. A preferred weight to weight ratio of ω-6 fatty acids having at least 18 carbon atoms to ω-3 fatty acids having at least 18 carbon atoms is 0.05-1, more preferably 0.1-0.6, most preferably 0.15-0.4.

The present composition preferably contains at least one oil selected from marine oils and eggs lipids. The marine oil is preferably selected from fish oil and algae oil. Preferably the present composition contains fish oil comprising DHA, EPA and preferably also DPA.

Saturated and Monounsaturated Fatty Acids

The present composition preferably comprises saturated and/or mono-unsaturated fatty acids. The amount of saturated fatty acids is preferably 6-60 wt. % based on total fatty acids, preferably 12-40 wt. %, more preferably 20-40 wt. % based on total fatty acids. In particular the amount of C14:0 (myristic acid)+C16:0 (palmitic acid) is preferably 5-50 wt. %, preferably 8-36, more preferably 15-30 wt. % wt. % based on total fatty acids. The total amount of monounsaturated fatty acids, such as oleic acid and palmitoleic acid, is preferably between 5 and 40 wt. %, more preferably between 15 and 30 wt. %. Including of the saturated and/or monounsaturated fatty acids provides an energy source, assisting the activities of prodromal subjects.

Phospholipids

Phospholipids are a source for choline and may prevent the decline in plasma choline levels after exercise. Choline is necessary for the formation of acetylcholine, a neurotransmitter involved in learning and memory and in the activation of muscles. These advantages are already achieved at relatively low phospholipid levels. Therefore, in addition to choline, the composition according to the invention may comprise one or more phospholipids.

Preferably, the present composition comprises one or more phospholipids, preferably 0.1-50 wt. % phospholipids based on total weight of lipids, more preferably 0.5-20 wt. %, more preferably between 1 and 5 wt. % based on total weight of lipids.

If used, the total phospholipid daily dosage is usually in the range of 50 to 5000 mg, in particular in the range of 100 to 2000 mg, more in particular in the range of 150 to 1200 mg.

As used herein, the term phospholipid includes lyso-phospholipids, de-acylated phospholipids and glycerophospholipids. In particular, the phospholipid is selected from the group of phosphatidylcholine (PC), phosphatidylethanolamine (PE), phosphatidylserine (PS), phosphatidic acid (phosphatidate), phosphoinositides (such as phosphatidylinositol (PI), phosphatidylinositol phosphate, phosphatidyl¬inositol bisphosphate, phosphatidylinositol triphosphate) and sphingomyelin. In a specific embodiment, the composition comprises at least two different phospholipids selected from the group consisting of phosphatidylserine, phosphatidylinositol, phosphatidylcholine and phosphatidylethanolamine. Lecithin, e.g. soy lecithin is a commercially available source of phospholipids. Milk fat can also be used as source of phospholipids, e.g. in the form of phospholipid concentrates.

Pyrimidine Derivative

A composition (for use) according to the invention comprises a pyrimidine derivative selected from the group of uridine sources and cytidine sources. These sources may be provided by any compound providing a uridine equivalent, respectively a cytidine equivalent, when administered to the human body. The term ‘ nucleoside equivalent’ (for nucleosides in general), respectively uridine equivalent or cytidine equivalent (for these specific nucleosides) is generally used in the art for compounds comprising a nucleobase, such as the nucleobase itself, nucleosides, mononucleotides (mono-, di- or triphosphates of nucleosides), oligonucleotides, polynucleotides and physiologically acceptable derivatives thereof that may be converted into the nucleoside as such or a nucleotide as such in vivo.

Examples of such derivatives include various esters. The acyl group may be any physiologically acceptable organic acid residue, in particular a C2-C24 organic acid residue. Preferred acylated forms of the pyrimidine sources are those wherein the (deoxy)ribose the has been acylated with acetic acid, n-caproic acid, caprylic acid, or n-capric acid, because these increase the bioavailability of the uridine or cytidine source. Methods for reacting these medium chain fatty acids to nucleosides, for example to the 5′ position of the nucleosides are known in the art per se and comprise conventional acylation methods. In a further embodiment the uridine source is acylated with a PUFA, for instance an omega-3 PUFA. WO 2002/088159 relates to uridine esters, which may be used in accordance with the present invention. The contents of this publication regarding (deoxy)uridine esters is incorporated by reference. Also (other) synthetic compounds can be suitably included as nucleoside source, e.g. acylated derivatives of the nucleosides, for example triacetyl-uridine.

Such equivalents are capable of increasing endogenous levels of the active forms of nucleosides in body, tissues such as blood, liver and brain. Useful ingredients include extracts of plant, animal, bacterial, algal or yeast material, as well as synthetic compounds.

The present composition preferably comprises uridine and/or an equivalent thereof, preferably at least one uridine or an equivalent thereof selected from the group consisting of uridine (i.e. ribosyl uracil), deoxyuridine (deoxyribosyl uracil), uridine phosphates (UMP, dUMP, UDP, dUDP, UTP, dUTP), nucleobase uracil, wherein optionally one or more hydroxyl moieties of the (deoxy)ribose of said nucleotide are acylated.

Suitable cytidine sources in particular include cytidine, deoxycytidine and derivatised (deoxy)cytidines. Derivatised de(oxidy)cytidines are preferably selected from the group of CMP, CDP, CTP dCMP, dCDP and dCTP, wherein optionally one or more hydroxyl moieties of the (deoxy)ribose of said nucleotide are acylated.

Preferably the present composition comprises an uridine phosphate selected from uridine monophosphate (UMP), uridine diphosphate (UDP and uridine triphosphate (UTP). Most preferably the present composition comprises UMP, as UMP is most efficiently being taken up by the body. Hence, inclusion of UMP in the present product enables a high effectively at the lowest dosage and/or the administration of a low volume to the subject. Preferably at least 50 wt. % of the uridine in the present composition is provided by UMP, more preferably at least 75 wt. %, most preferably at least 95 wt. %. The present composition is preferably used in a method comprising the administration of the pyrimidine derivative (the cumulative amount of uridine sources and cytidine source) in an amount of 0.08-3 g per day, preferably 0.1-2 g per day, more preferably 0.2-1 g per day.

The use of the composition preferably comprises the administration of a composition providing a uridine source in a concentration of 0.4 mg-3000 mg, calculated as UMP, per 100 kcal, preferably 0.6 mg-2000 mg, calculated as UMP, per 100 kcal product, more preferably −1 mg-1000 mg, calculated as UMP per 100 kcal product.

Preferably 1-37.5 mg, uridine equivalent, calculated as UMP, per kilogram body weight is administered per day. The required dosages of the equivalents on a weight base can be calculated from the dose for UMP by taking equimolar amounts using the molecular weight of the equivalent and of UMP, the latter being 324 Dalton.

If present, the content of the cytidine source(s) is usually between 0.4 and 3000 mg, calculated as cytidine monophosphate, per 100 kcal of the composition.

The pyrimidine derivatives selected from uridine sources and cytidine sources are preferably present in a composition according to the invention an amount providing—in total—at least 1.2 mol/100 kcal composition, more preferably 1.2 mol/100 kcal-9.26 mmol/100 kcal composition, more preferably 1.8 mol/100 kcal-6.173 mmol/100 kcal, more preferably 3 mmol/100 kcal-3.1 mmol/100 kcal of said pyrimidine derivative(s) (uridine/cytidine) units.

Although cytidine is a precursor of uridine, it is more efficient and effective to include one or more uridine equivalents in the present composition. Accordingly, it is preferred that 60-100 wt. %, in particular 90-100% of the pyrimidine derivative content is formed by one or more uridine sources.

Methyl Donors

Due to the protein deficient diet of PKU patients, there is a potential decreased intake of methyl donors that are often present in e.g. meat, fish and dairy products that cannot be consumed in sufficient quantities by PKU patients. Therefore, the present composition preferably contains significant amounts of methyl donors.

Methyl donors are those food grade compounds which are capable of providing a methyl, methylene or formyl group when administered to a human individual in vivo. The methyl donor included in the present composition is preferably selected from serine, methionine, choline, betaine, dimethylglycine and sarcosine and derivatives thereof. The methyl donors can be included in the formula as pure compounds as such, as their salts and as compounds, wherein the methyl donor is covalently bound to amino acids, and which have a molecular weight less than 600 Dalton.

Choline refers to the various quaternary ammonium salts containing the N,N,N-trimethylethanolammonium cation. More specifically, choline is selected from the group of the choline cation, choline salts or esters, such as choline chloride, choline bitartrate, choline stearate, or the like, or compounds that dissociate to choline, such as choline alfoscerate, sphingomyelin, cytidine-diphospho-choline or citicoline or CDP-choline, acylglycerophosphocholines, e.g, lecithin, lysolecithin, glycerophosphatidylcholine, and any mixture thereof. Preferably the present composition contains a choline salt, in particular choline chloride, and/or phosphatidylcholine.

The present method preferably comprises the administration of more than 50 mg choline per day, more preferably 80-2000 mg choline per day, more preferably 120-1000 mg choline per day, most preferably 150-600 mg choline per day. The present composition (for use) according to the invention preferably comprises 10-2000 mg choline per 100 kcal. Preferably at least 10 mg/100 kcal, more preferably at least 20, 30, 40, 50, 60, 70, 80, 90, 100 mg/100 kcal. The preferred upper limit for the above mentioned lower limit is 2000 mg/100 kcal, more preferably 1750, 1500, 1250 or 1000 mg/100 kcal.

The total daily dose of methyl donors can be calculated by taking equimolar amounts as defined for choline and correcting for the molecular weight of that methyl donor. The total daily dose is preferably at least 0.48 mmol methyl donor per day, more preferably at least 0.77 mmol per day, more preferably at least 1.15 mmol per day, most preferably at least 1.22 mmol per day. The total daily dose preferably is 20 mmol or less, more preferably 10 mmol or less, most preferably 10 mmol or less. In case the composition is a liquid product, the present composition preferably comprises 0.48-30 mmol methyl donor per 100 ml, preferably 1.9-10 mmol methyl donor/100 ml.

As shown in Examples 1 and 2, PKU patients are likely to benefit from the inclusion of the selected methyl donors.

Vitamin B

In an embodiment, the composition (for use) according to the invention comprises at least one vitamin B selected from the group of vitamin B6, vitamin B9 and vitamin B12. Vitamin B6 includes pyridoxine, pyridoxal, pyridoxamine, and pyridoxine salts, e.g. the hydrochloride or phosphate salt. Vitamin B9 is also known as folic acid or folate. Vitamin B12 is also known as cobalamines.

In particular, good results have been achieved with a combination comprising vitamin B6, vitamin B12 and vitamin B9.

It should be noted that one or more other vitamins of the vitamin B family may be present in a composition (for use) according to the invention. Such other vitamins B include in particular vitamin B1 (thiamine), vitamin B2 (riboflavin), vitamin B3 (niacin or niacinamide), vitamin B5 (pantothenic acid), and vitamin B7 (biotin).

The vitamin B is to be administered in an effective dose, which dose depends on the type of vitamin B used. As a rule of thumb, a suitable minimum or a maximum dose may be chosen based on known dietary recommendations, for instance as recommended by Institute of Medicine (IOM) of the U.S. National Academy of Sciences or by Scientific Committee on Food (a scientific committee of the EU), the information disclosed herein and optionally a limited amount of routine testing. A minimum dose may be based on the estimated average requirement (EAR), although a lower dose may already be effective. A maximum dose usually does not exceed the tolerable upper intake levels (UL), as recommended by IOM.

If present, the vitamin B6 is usually present in an amount to provide a daily dosage in the range of 0.5 to 10 mg, in particular in the range of 0.75 to 5 mg, more in particular in the range of 0.9 to 2.5 mg If present in the composition according to the invention, the vitamin B6 concentration is usually 0.05-10 mg Vitamin B6 per 100 kcal, preferably between 0.1-1 mg/100 kcal. If present, the Vitamin B12 content in a composition (for use) according to the invention usually is between 0.05-5 μg Vitamin B12/100 kcal, preferably between 0.1 and 2.5 μg/100 kcal, and even more preferably between 0.2 and 2.0 μg/100 kcal. If present, the vitamin B9 is usually present in an amount to provide a daily dosage in the range of 50 to 5000 μg, in particular in the range of 150 to 1000 μg, more in particular in the range of 200 to 1000 μg.

(v) Anti-Oxidant

The composition according to the invention may comprise an antioxidant selected from the group of vitamin C, vitamin E and selenium. The vitamin C may be present as free acid (ascorbic acid) or as a salt, e.g. sodium ascorbate or potassium ascorbate. Suitable sources of vitamin E include (alpha-)tocopherol and tocotrienol. Suitable sources of selenium include selenate and selenite.

The anti-oxidant is to be administered in an effective dose. As a rule of thumb, a suitable minimum or a maximum dose may be chosen based on known dietary recommendations, for instance as recommended by the Institute of Medicine (TOM) of the U.S. National Academy of Sciences or by the Scientific Committee on Food (a scientific committee of the EU), the information disclosed herein and optionally a limited amount of routine testing. A minimum dose may be based on the estimated average requirement (EAR), although a lower dose may already be effective. A maximum dose usually does not exceed the tolerable upper intake levels (UL), as recommended by IOM. If present in the combination, vitamin C is usually present in an amount to provide a daily dosage in the range of 20 to 1200 mg, in particular in the range of 30 to 400 mg, more in particular in the range of 35 to 120 mg. If present in the nutritional or pharmaceutical composition according to the invention, the vitamin E is usually present in an amount to provide a daily dosage in the range of 8 to 200 mg, in particular in the range of 20 to 140 mg, more in particular in the range of 35 to 100 mg.

If present in the nutritional or pharmaceutical composition according to the invention, the selenium is usually present in an amount to provide a daily dosage in the range of 40 to 400 μg, in particular in the range of 50 to 200 μg, more in particular in the range of 55 to 80 μg.

Optionally one or more antioxidants may be present other than (v) the antioxidant selected from the group of vitamin C, vitamin E and selenium.

Further Components

Furthermore, the composition (for use) according to the invention may comprise one or more further micronutrients, for instance one or more micronutrients selected from the group of (further) vitamins, minerals, and trace elements, taurine and inositol. In particular, in an advantageous embodiment the composition comprises vitamin A and/or vitamin D. In addition, the composition may comprise dietary fibre.

Nutritional Composition

Compositions according to the claimed invention are preferably nutritional compositions comprising carbohydrate, lipid and protein. Preferably the composition further comprises vitamins, choline, antioxidant and/or minerals (in particular as identified above). Preferably vitamin A and D, that are often supplied by normal protein sources, like dairy or meat are contained in the nutritional composition according to the claimed invention, the lipid comprises polyunsaturated fatty acids selected from the group consisting of EPA, DHA, and DPA. The nutritional composition according to the claimed invention further comprises a pyrimidine derivative selected from uridine sources and cytidine sources, preferably uridine and/or an equivalent thereof, preferably at least one uridine or an equivalent thereof selected from the group consisting of uridine (i.e. ribosyl uracil), deoxyuridine (deoxyribosyl uracil), uridine phosphates (UMP, dUMP, UDP, UTP), nucleobase uracil and acylated uridine derivatives, most preferably uridine mono phosphate.

In a specific embodiment, the nutritional composition provides a complete nutrition, i.e. nutrition providing vitamins, minerals, and food energy in the form of carbohydrates, proteins, and fats in suitable amounts to provide a healthy nutritional intake. Advantageously the complete nutrition also contains dietary fibre and/or a probiotic. Suitable compositions for complete nutritions are known in the art, and the present nutritional composition can be based on Foods for Special Medical Purposes regulations (FSMP, EC, http://eur-lex.europa.eu/legal-content/EN/ALL/?uri=CELEX:31999L0021), with the proviso that the nutritional composition (for use) according to the invention comprises one or more of said pyrimidine derivatives and one or more of said ω-3 fatty acids.

Treatment of PKU Patient

A composition (for use) in accordance with the invention, in particular a nutritional composition, is in particular suitable for treatment of a PKU patient. It can be used to decrease the Phe level in the blood of patient when it is above a desired concentration, or close to a maximally desired concentration. Thus, the composition can also be used to normalize the Phe concentration to a normal (non-pathological) level. The invention is however also very useful for use in the nutritional management (dietary management) of Phe levels in the blood of a PKU patient. The goal of nutritional management for those with PKU is to maintain plasma phe concentrations that support healthy growth, development, and mental functioning while providing a nutritionally complete diet. Erin L. MacLeod and Denise M. NeyAnn describe how nutritional management of PKU patients can be carried out in Nestlé [Engl] 2010; 68:58-69. The subject to be treated can in principle any PKU patient, although the invention is in particular advantageous for a PKU patient who has an elevated Phe level, more in particular a level that is toxic, or for treatment of a PKU patient who is not generally complying to a Phe-free or Phe-restricted diet, and thereby has an increased risk of being exposed to too high amounts of Phe.

A lowest Phe level in blood that is considered as toxic depends to a certain extent on the patient, in particular the patient's age. Further, some differences exist between different countries on recommended guidelines for the Phe level in blood. Generally, a level of about 1500 μmol/l in blood is considered toxic, but toxicity could already occur at considerably lower levels, amongst others, dependent on factors like the age of the patient.

The following Table shows recommended Phe levels in blood for different patient groups in some countries.

Blood phe level, μM

| Age | UK | Germany | France | US |
|---|---|---|---|---|
| Birth¹ | >400 | >600 | >600 | >600 |
| Infant to 10 | 120-360 | 40-240 | 120-360 | 120-360 |
| years | | | | |
| 10-12 years | <480 | <600 | <900 | 120-360 |
| 12-20 years | <480 | <600 | <900 | 120-600 |
| >20 years | <700 | <1,200 | <900 | 120-900 |
| Pregnancy | | | | 120-360 |

Normalisation of a Phe level in blood in accordance with the invention refers in particular to reducing the Phe concentration from a value above a highest recommended level, to a concentration within a recommended range. Dietary management is aimed at maintaining the Phe concentration in blood within a recommended range.

Typically, the composition is for use in decreasing the phenylalanine level in the blood to, normalizing the phenylalanine level in the blood to or maintaining the phenylalanine level in the blood at a concentration below 1200 μM, in particular below 900 μmol/l, more in particular below 600 μmol/l.

Generally, a treatment with a composition (for use) according to the invention of a human child having an age of 0-10 years is aimed at lowering to, normalising to or maintaining at a phenylalanine concentration in the blood of the patient in the range of 40-360 μmol/l, preferably 120-360 μmol/l.

Generally, a treatment with a composition (for use) according to the invention of a human child having an age of 10-12 years is aimed at lowering to, normalising to or maintaining at a phenylalanine concentration in the blood of the patient to a concentration below 900 μmol/l, in particular below 600 μmol/l, preferably below 480 μmol/l, more preferably 120-360 μmol/l.

Generally, a treatment with a composition (for use) according to the invention of a human having an age 12-20 years of age is aimed at lowering to, normalising to or maintaining at a phenylalanine concentration in the blood of the patient to a concentration below 900 μmol/l, in particular below 600 μmol/l, preferably below 480 μmol/l, more preferably in the range of 120-600 μmol/l.

Generally, a treatment with a composition (for use) according to the invention of a human having an age of more than 20 years is aimed at lowering to, normalising to or maintaining at a phenylalanine concentration in the blood of the patient to a concentration below 1200 μmol/l, in particular below 900 μmol/l, in particular below 700 μmol/l, more in particular 600 μmol/l or less. The lower limit of the range can be 0, in particular 120 μmol/l.

In case of pregnancy, the treatment is generally aimed at lowering to, normalising to or maintaining at a phenylalanine concentration in the blood of the patient to a concentration in the range of 120-360 μmol/l.

Although, also when treated with a composition (for use) according to the invention it is advisable to restrict the daily intake of Phe to a lower daily intake than is generally recommended for subjects that are not suffering from PKU, the present invention reduces the risk of adverse effects of consumption of a food product containing a significant amount of Phe.

The composition is usually administered orally, although another administration mode, e.g. tube feeding or as a suppository, is feasible.

Next, the invention is illustrated by a number of Examples.

### Example 1 Phe Lowering Effect of Nutritional Product in PKU Mouse Model (C57Bl/6 Pahenu2)

Material and Methods

Mouse Model

In this experiment, the C57Bl/6 Pahenu2 mouse model was used. Male and female homozygous (PKU) mice and wild-type (WT) littermates were obtained from our own breeding. Male and female mice were housed in separate rooms under the same 12:12 light/dark cycle, temperature and humidity conditions. 60 PKU individuals and 10 WT mice were subdivided into seven experimental groups, receiving different diets. The basal formula for all diets was AIN93G (Research Diet Services, Wijk bij Duurstede).

Diets

The WT individuals received normal chow with a Phe content of 8.8 g/kg. The six groups of PKU mice arisen when the supplementation of Fortasyn Connect® (N.V. Nutricia) ingredients and three different Phe contents was randomized. Fortasyn Connect® (FC), is a combination of nutrients including antioxidants (Selenium, vitamin E), uridine-5′-monophosphate, choline, vitamins B12 and B6, folic acid, phospholipid, docosahexaenoic acid (DHA) and eicosapentaenoic acid.

This resulted in the following groups. The first two groups received the same AIN93G normal chow as previously described for WT individuals (Phe content of 8.8 g/kg). One of these groups received the diet without FC (control), the other received it with FC, these groups were called respectively: ‘Phe8.8’ and ‘Phe8.8+FC’. The control group received lower amounts of Se, VitE, Choline, Vit. B12, B6 en folic acid compared to FC group. In addition the FC group received EPA/DHA, UMP and phospholipid (lecithin) while control groups were not supplemented with these ingredients. Two groups received a low-Phe diet. This diet contained 4.0 g/kg Phe without or with FC, respectively called ‘Phe4.0’, and ‘Phe4.0+FC’. Finally, two mid-Phe content groups were created wherein the Phe levels sets were set in between the high-Phe and Low Phe groups. The content of the mid-Phe diet was 6.4 g/kg, without FC (Phe6.4) and with FC (Phe6.4+FC). All diets met the minimal nutritional requirement for laboratory animals. The animals had ad libitum access to these diets and water for 12 weeks. At the start of the experiment (post-natal day (PND) 28), the animals were weaned and a blood sample was taken by tail vein puncture. On PND 31, the animals were exposed to a circular open field arena and subsequently put on the different diets for 12 weeks. All animals were weighed daily between 16:00-18:00 in the first week of the experiment (PND 31-PND 38) and from then on weekly. Food intake was measured daily. Within these 12 weeks, blood collection was performed on PND 59 and 87. On PND 115, were euthanized via a heart puncture to collect blood, followed by perfusion. All proceedings concerning animals within this study were approved by the ethical committee of the University of Groningen, The Netherlands.

Blood Collection

Blood samples were taken with three different techniques. On PND 28, a small piece of tail tissue was collected for genotyping. Immediately after this procedure, a blood samples was collected from the wound at the tip of the tail. On PND 59 and 87, blood samples were collected via tail vein sampling. In both techniques, Na-heparinized micro haematocrit tubes (VITREX, ISO 12772, REF 161313) were used to collect blood before transferring the blood to filter paper. On PND 115, the animals were anesthetized with an intraperitoneal (ip) injection of pentobarbital. When hind-paw reflex was no longer present, a heart punction was performed. Collected blood was stored in a micro tube prepared with lithium-heparin (Sarstedt, REF 41.1393.005) before centrifuging the blood samples at 4° C. for 10 min at 12800 rpm. Blood plasma was collected and stored at −80° C. before analysis.

Amino Acid Analysis

In dried blood spots, phenylalanine and tyrosine were quantified using high-performance liquid chromatography tandem mass spectrometry. Blood was collected on filter paper and air dried for at least 24 hours. A pinch was taken with a diameter of 4 mm of dried blood spot and left to elute in methanol, containing deuterated phenylalanine and tyrosine as internal standards. Liquid chromatography was performed using a Nexera LC30 (Shimadzu, Kyoto, Japan). Mass spectrometry was performed using an API-3000 (AB-Sciex, Framingham, USA).

To measure amino acid (except for tryptophan) in whole plasma, samples were vortexed and centrifuged at 20.800 rcf for 4 min. After centrifuging, 85 μl of the supernatant was pipetted into capsules. Norleucine in sulfosalicylic acid was added as an internal standard (1:1 v/v). Amino acid concentrations were measured by high-performance liquid chromatography (HPLC) followed by post-column ninhydrin derivatization on a Biochrom 30 analyser (Pharmacia Biotech, Cambridge, UK).

Statistical Analysis

Blood phenylalanine values were tested for normality with the Shapiro-Wilk normality test. Only WT individuals and Phe4.0 groups did not have normal distributed values. Despite these findings, a One-way ANOVA was executed and a Bonferonni was used as a post-hoc test. Phenylalanine intake values were also tested for normality with the Shapiro-Wilk normality test. WT individuals showed a non-normal distribution (p=0.0482). While all other groups did show a normally distributed data, we performed a One-way ANOVA with a Bonferonni post-hoc test. Correlations between absolute Phe-intake and Phe levels in the blood were examined with a Pearson's correlation. With an Fisher r-to-z transformation, a significant differences between correlation were explored.

Key in the pathophysiology of the PKU is raised Phe levels in the blood. Current treatment in the Netherlands of PKU is characterised by managing these levels between the 120 μmol/l and 360 μmol/l for children (0-12 years) and between 120 μmol/l and 600 μmol/l from 12 years on (Left untreated, Phe levels could raise to 1000 and 2000 μmol/l. As patients often have difficulties maintaining the diet, the levels could be anywhere between the previous mentioned levels. In this current experiment different Phe-content was offered to the animals to mimic, to some extent, these different groups of patients.

Results in FIGS. 1A-1E show that Phe levels in blood decrease with decreasing Phe intake, but surprisingly the test groups in the two groups with the highest Phe intake (Phe 8 and Phe 6) were statistically significant lower than the placebo group. This shows that the additional ingredients in the FC group are able to lower the Phe levels in the blood.

Phe levels in blood are depicted in FIG. 1B and a one-way ANOVA revealed significant differences between Phe8.8 and Phe8.8+FC and Phe6.4 and Phe6.4+FC in the PKU mice, as indicated with asterisks.

### Example 2 Phe Lowering Effect of Nutritional Product in PKU Mouse Model (BTBR PKU Mice)

In this second set of experiments, the same procedure was performed as in Example 2 with the following modifications: the mice that were used had the same mutation (Pahenu2) but on a BTBR genetic background instead of C57Bl/6. In addition, there were 6 groups now: wild-type (WT) with normal chow, WT+FC, PKU with normal chow (Phe 6.2), PKU with normal chow+FC, PKU with low Phe (2.2) and PKU with low Phe+FC.

At four months of age, blood was collected via tail vein sampling into Na-heparinized micro haematrocrit tubes (VITREX, ISO 12772, REF 161313) before transferring the blood to filter paper and analyzing Phe levels like described above.

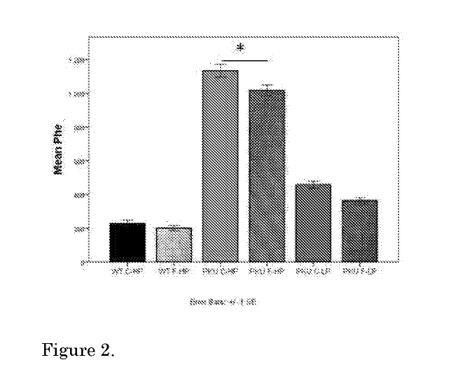

Phe levels in blood are depicted in FIG. 2 and a one-way ANOVA revealed a significant difference between high Phe and high Phe+FC in PKU mice, as indicated with an asterisk.

### Example 3 Exemplary Composition (for Use) According to the Invention

TABLE 2

| | invention formula |
|---|---|
| | (per serving) |
| Energy (kcal) | 173.5 |
| Protein/Protein Equivalent (g) | 20 |
| Carbohydrates (g) | 16.5 |
| Fat (g) | 3.05 |
| DHA (22:6n-3; mg) | 0.88 |
| Micronutrients* | |
| Calcium (mg) | 356 |
| Phosphorus (mg) | 276 |
| Vitamin E (mg α-TE) | 33.6 |
| Vitamin C (mg) | 44.4 |
| Vitamin D (μg) | 6 |
| Vitamin K (μg) | 20 |
| Folic acid (μg) | 160 |
| Niacin/Vitamin B3 (mg) | 7.1 |
| Vitamin B6 (mg) | 0.58 |
| Vitamin B12 (μg) | 1.8 |
| Choline (mg) | 153 |
| Nucleotides | |
| Uridine-5′-monophosphate | 625 |
| (mg) | |

1. A composition comprising (i) one or more ω-3 fatty acids selected from the group consisting of DHA, DPA and EPA and (ii) one or more pyrimidine derivatives selected from the group consisting of uridine sources and cytidine sources, said composition in a daily dose amount to decrease or normalize the phenylalanine level in the blood of a PKU patient or to manage the phenylalanine level in the blood of a PKU patient.

2. The composition according to claim 1, wherein the composition further comprises a choline and/or a phospholipid.

3. The composition according to claim 2, wherein the composition is to be administered in an amount providing a daily dose of 0.1-5 g choline.

4. The composition according to claim 1, wherein the composition further comprises protein and 0-10 mg phenylalanine per g protein.

5. The composition according to claim 1, wherein the composition is a nutritional composition comprising lipid, digestible carbohydrates, protein in concentrations based on the dry weight of the composition of between 1-15 wt % lipid, 5-60 wt % carbohydrate, and 1-40 wt % protein, and further comprising vitamins and minerals according to the FSMP regulations as laid down in EC Commission Directive 1999/21/EC.

6. The composition according to claim 1, wherein the composition comprises between 0.1-40 wt % DHA based on total lipid content of the composition and between 0.4 and 3000 mg uridine source, calculated as uridine monophosphate, per 100 kcal of the composition.

7. The composition according to claim 1, further comprising at least one vitamin B selected from the group consisting of vitamin B6, vitamin B12 and vitamin B9.

8. The composition according to claim 1, further comprising an anti-oxidant selected from the group consisting of vitamin C, vitamin E and selenium.

9. A method of decreasing the phenylalanine level in the blood of a human PKU patient, normalizing the phenylalanine level in the blood of a human PKU patient or maintaining the phenylalanine level in the blood of a human PKU patient comprising administering the composition of claim 1 to the human PKU patient wherein the phenylalanine level is decreased, normalized or maintained at a concentration below 1200 μmol/l.

10. The method according to claim 9, wherein the PKU patient is a human child having an age of 0-10 years and the phenylalanine level in the blood is to be lowered to, to be normalized to or to be maintained at a concentration in the range of 40-360 μmol/l.

11. The method according to claim 9, wherein the PKU patient is a human having an age of 10-20 years and the phenylalanine level in the blood is to be lowered to, to be normalized to or to be maintained at a concentration of 600 μmol/l or less.

12. The method according to claim 9, wherein the PKU patient is a human having an age 20 years or more and the phenylalanine level in the blood is to be lowered to, to be normalized to or to be maintained at a concentration of less than 1200 μmol/l.

13. The method according to claim 9, wherein the PKU patient is pregnant and the phenylalanine level in the blood is to be lowered to, to be normalized to or to be maintained at a concentration in the range of 120-360 μmol/l.

14. The method of claim 9 wherein the composition is to be administered orally.

15. A nutritional composition comprising protein, lipid and carbohydrates wherein the protein provides at least all essential amino acids other than phenyl alanine, and optionally phenylalanine, which—if present—is present in a phenylalanine concentration of 0-10 mg/per gram protein; the lipid comprising one or more ω-3 fatty acids selected from the group consisting of DHA, DPA and EPA in a total concentration of at least 0.05 wt % based on total lipid content; and one or more pyrimidine derivatives selected from the group consisting of uridine sources and cytidine sources providing between 0.4 and 3000 mg uridine source, calculated as uridine monophosphate per 100 kcal, of the composition and/or between 0.4 and 3000 mg cytidine source, calculated as cytidine monophosphate, per 100 kcal of the composition.

16. The nutritional composition according to claim 15, wherein the composition comprises the one or more pyrimidine derivatives in a total content providing 0.001-9.26 mmol/100 kcal.

17. The nutritional composition according to claim 15, wherein the composition comprises 1-15 wt. % lipid, 5-60 wt. % digestible carbohydrate and 1-14 wt. % protein, all based on the dry weight of the composition.

18. The nutritional composition according to claim 15, wherein the composition comprises a choline.

19. The nutritional composition according to claim 15, comprising
i) DHA and EPA
ii) uridine and/or uridine monophosphate
iii) at least one vitamin B selected from the group consisting of vitamin B6, vitamin B9 and vitamin B12
iv) phosphatidylcholine and/or one or more other phospholipids; and
v) an antioxidant selected from the group consisting of vitamin C, vitamin E and selenium.

20. The nutritional composition according to claim 19, wherein the composition comprises vitamin B6, vitamin B12, vitamin B9, vitamin C, vitamin E, selenium and at least one choline selected from the group consisting of choline chloride and choline alfosclerate.

21. The nutritional composition according to claim 15, wherein the composition comprises at least two different phospholipids selected from the group consisting of phosphatidylserine, phosphatidylinositol, phosphatidylcholine and phosphatidylethanolamine.

22. The nutritional composition according to claim 15, comprising vitamin A and/or vitamin D.

23. (canceled)
